SEQUENCE LISTING (21 sequences)

SEQ ID NO: 1 - protein, 25 aa, Homo sapiens
HSDAFTDNYTRLRKQAKKYLNSILN

SEQ ID NO: 2 - protein, 25 aa, Homo sapiens
HSDAFTDNYTRLRKQAKKYLNSILN

SEQ ID NO: 3 - protein, 11 aa, Hono sapiens
AVKKYLNSILN

SEQ ID NO: 4 - protein, 4 aa, Homo sapiens
KKYL

SEQ ID NO: 5 - protein, 3 aa, Homo sapiens
KKY

SEQ ID NO: 6 - protein, 5 aa, Homo sapiens
VKKYL

SEQ ID NO: 7 - protein, 6 aa, Homo sapiens
AVKKYL

SEQ ID NO: 8 - protein, 5 aa, Homo sapiens
NSILN

SEQ ID NO: 9 - protein, 4 aa, Homo sapiens
KKYV

SEQ ID NO: 10 - protein, 4 aa, Homo sapiens
SILN

SEQ ID NO: 11 - protein, 5 aa, Homo sapiens
NSYLN

SEQ ID NO: 12 - protein, 5 aa, Homo sapiens
NSIYN

SEQ ID NO: 13 - protein, 3 aa, Homo sapiens
AVK

SEQ ID NO: 14 - protein, 3 aa, Homo sapiens
KYL

SEQ ID NO: 15 - protein, 3 aa, Homo sapiens
KKY

SEQ ID NO: 16 - protein, 5 aa, Homo sapiens
AVKKY

SEQ ID NO: 17 - protein, 5 aa, Homo sapiens
VKKYL

SEQ ID NO: 18 - protein, 5 aa, Homo sapiens
LNSIN

SEQ ID NO: 19 - protein, 6 aa, Homo sapiens
YLNSIN

SEQ ID NO: 20 - protein, 4 aa, Homo sapiens
KKYL

SEQ ID NO: 21 - protein, 5 aa, Homo sapiens
NSILN

(19) United States
(12) Patent Application Publication
(10) Pub. No.: US 2005/0075284 A1
(43) Pub. Date: Apr. 7, 2005

(54) Pharmaceutical compositions comprising vip-related peptides for the treatment of sexual disorders

(76) Inventors: Illana Gozes, Hasharon (IL); Matityahu Fridkin, Rehovot (IL)

Correspondence Address: NATH & ASSOCIATES, 1030 15th STREET, NW, 6TH FLOOR, WASHINGTON, DC, 20005, US

(21) Appl. No.: 10/240,392

(22) PCT Filed: May 22, 2001
(86) PCT No.: PCT/IL01/00460

(51) Int. Cl. A61K 38/22

(52) U.S. Cl. 514/12

(57) ABSTRACT

The present invention relates to pharmaceutical compositions for the treatment of female sexual dysfunction, for vaginal relaxation and/or for modulation of sperm motility. The composition comprises as active ingredient (i) a peptide analogue or conjugate of vasoactive intestinal peptide (VIP) as defined in the specification and is preferably formulated for topical application in the vaginal, vulvar and/or clitorial area. The invention also relates to the use of the VIP peptide analogue or conjugate for the preparation of a pharmaceutical composition for the treatment of female sexual dysfunction, for vaginal relaxation and/or for modulation of sperm motility. Yet further, the invention provides a method of treatment of female sexual dysfunctions and/or vaginal relaxation by the use of said peptide analogue and/or conjugate.

### FIELD OF THE INVENTION

The present invention relates to certain peptides, particularly analogues and fragments of vasoactive intestinal peptide (VIP), and conjugates thereof with lipophilic groups, for the treatment of female sexual disorders.

### BACKGROUND OF TE INVENTION

Sexual dysfunction is defined as any of a group of sexual disorders characterized by inhibition either of sexual desire or of the psychophysiological changes that usually characterize sexual response such as male erectile disorder and female sexual arousal disorder.

Sexual dysfunctions are disturbances in the sexual response cycle or pain associated with sexual arousal or intercourse. Proper sexual functioning in men and women depends on the sexual response cycle, which consists of an anticipatory mental set (sexual motive state or state of desire), effective vasocongestive arousal (erection in men; swelling and lubrication in women), orgasm and resolution.

The sexual response cycle is mediated by a delicate, balanced interplay between the sympathetic and parasympathetic nervous systems. Vasocongestion is largely mediated by parasympathetic (cholinergic) outflow; orgasm is predominantly sympathetic (adrenergic). These responses are easily inhibited by cortical influences or by impaired hormonal, neural or vascular mechanisms.

Disorders of sexual response may involve one or more of the cycle's phases. Generally, both the subjective components of desire, arousal and pleasure and the objective components of performance, vasocongestion, and orgasm are disturbed, although any may be affected independently.

Sexual dysfunction may be lifelong or acquired and may have a psychologic, physiologic or combined etiology. Thus, some diseases such as diabetes mellitus, cancer, neurologic diseases, etc. and/or some drugs such as alcohol, antihypertensives, sedatives, opioids, etc. may cause sexual dysfunction.

The most common types of sexual dysfunction in women are hypoactive sexual desire disorder, sexual arousal disorder and dyspareunia (painful coitus). In addition to the above-mentioned diseases and drugs that affect the sexual activity in both sexes, women are affected also by pelvic disorders such as endometriosis, cystitis and vaginitis, by estrogen deficiency and by oral contraceptives.

Among the methods commonly used for treatment of sexual dysfunction in women are estrogen therapy and combined estrogen/progestogen therapy. However, in view of the increased risk of endometrial cancer with estrogen therapy and the androgenic activity of progestogens, there is a need for alternative therapies for the treatment of sexual dysfunction in women. U.S. Pat. No. 5,877,216, herein incorporated by reference in its entirety as if fully disclosed herein, describes a method for treating sexual dysfunction in women by administration to the vagina and/or vulval area of a pharmaceutical formulation containing a selected vasodilating agent such as a prostaglandin.

Vasoactive intestinal peptide (VIP) release may induce physiological changes in sexual arousal and excitement, and may be the major neurotransmiter that participates in the innervation of the vaginal blood supply, including small blood vessels, smooth mucle and epithelial cells in the vaginal tract.

VIP has been proposed for induction of vaginal lubrication in female mammals, as described in International PCT Publication No. WO 88/03928 and corresponding Australian Patent No. 609765, both herein incorporated by reference in their entirety as if fully disclosed herein. According to this disclosure, VIP is administered locally by injection to the inner wall of the vagina, or systemically by intravenous injection or by continuous infusion.

Systemic administration of VIP to females has been found to decrease uterine smooth muscle activity and increase vaginal blood flow (Ottesen et al., Eur. J. Clin. Invest. 13, 321-324, 1983). In the male, VIP induces penile erection and was suggested to induce clitorial arousal in the female (Hauser-Kronberger et al., Peptides 20, 539-543, 1999).

The present inventors have synthesized VIP derivatives, including VIP analogues and fragments, and conjugates thereof with lipophilic groups, for use in the treatment of male sexual dysfunction/impotence and for the treatment of neurodegenerative disorders (Gozes and Fridkin, J. Clin. Invest. 90, 810-814, 1992; Gozes et al., Endocrinology, 134,2121-2125,1994; Gozes et al., J. Pharmacol. Exper. Therap. 273, 161-167, 1995; Gozes et al., Proc. Natl. Acad. Sci. USA 93, 427-432, 1996; Gozes et al., Proc. Natl. Acad. Sci. USA 96, 41434148, 1999, and U.S. Pat. No. 5,147,855, U.S. Pat. No. 5,998,368, EP 620008, and PCT Publication WO 97/40070, all these documents being herein incorporated by reference in their entirety as if fully disclosed herein). These derivatives may distinguish between different VIP receptors (ibid 1995) and enhance cGMP formation. One of these derivatives, a stearoyl (St) conjugate of an analogue of VIP wherein the methionine (Met) residue at the 17-position of VIP was replaced by a norleucine (Nle) residue (herein, St-Nle¹⁷-VIP or SNV), was shown to be 1000-fold more potent than VIP in enhancing cGMP formation (Ashur-Fabian et al., Peptides 20, 629-633, 1999).

Another peptide (VIP-related) found in the urogenital tract, pituitary-adenylate cyclase activating polypeptide (PACAP), seems to be involved in stimulation of sperm motility (Gozes et al., Ann. NY Acad. Sci. USA, 865, 266-273, 1998), suggesting an added value of female sexual stimulation with VIP-PACAP-related compounds. Indeed, the short VIP fragment analogue Lys-Lys-Tyr-Leu (ibid Proc. Natl. Acad. Sci. USA, 1999) is a sequence shared by VIP and PACAP.

The limited treatment procedures for female sexual dysfunction and the side effects of these therapies strengthen the need for additional therapies.

### SUMMARY OF THE INVENTION

The present invention provides, in one aspect, a pharmaceutical composition for treatment of female sexual dysfunction and/or for vaginal relaxation, comprising a pharmaceutically acceptable carrier and as active ingredient a VIP-related peptide selected from the group consisting of:
(i) a peptide analogue of vasoactive intestinal peptide (VIP) in which one or more amino acids has been replaced, added or deleted without substantially altering the biological properties of the parent peptide;
(ii) a conjugate of VIP or of a peptide analogue of (i) coupled to a lipophilic moiety;
(iii) a physiologically active fragment of VIP, or of a peptide analogue (i) or of a conjugate (ii); and (iv) a functional derivative of any of (i), (ii) and (iii).

In this aspect of the invention, the pharmaceutical composition of the invention is suitable for improving vaginal muscle tone and vaginal tissue health, for enhancing vaginal lubrication and enhancing sexual arousal. These activities of the VIP-related peptides will also prevent vaginal and vulvar inflammations and infections, such as bacterial, fungal and other infections of the genital tract of a female associated with insufficient vaginal lubrication activity, particularly in postmenopausal women.

In addition, the pharmaceutical composition of the invention is suitable for the treatment of vaginal relaxation, particularly, excessive vaginal relaxation.

In another aspect, the invention relates to a pharmaceutical composition for modulation of sperm motility comprising a VIP-related peptide as defined above. According to this aspect, the VIP-related peptide may be designed to be used in vivo such as to increase sperm motility and thus to enhance copulation efficiency and to promote conception, or it may be designed to reduce sperm motility and thus to prevent conception. In another embodiment, the VIP-related peptide may be used in vitro to enhance sperm motility in in-vitro fertilization procedures.

In both aspects of the invention performed in vivo, the pharmaceutical composition is preferably for topical application in the vaginal, vulvar and/or clitorial area, in the form of a cream, gel, suspension, ointment, solution, foam or liposomal composition.

The VIP-related peptide used in the pharmaceutical composition is any of the peptides and conjugates described in U.S. Pat. No. 5,147,855, U.S. Pat. No. 5,998,368, EP 620008, and PCT Publication WO 97/40070, all these documents being herein incorporated by reference in their entirety as if fully disclosed herein.

In one embodiment, the VIP-related peptide is an analogue of VIP in which one or more amino acids has been replaced, of the sequence (SEQ ID NO: 1):

1 7
His-Ser-Asp-Ala-X¹-Phe-Thr-Asp-Asn-Tyr-Thr-Arg-
16 28
Leu-Arg-Lys-Gln-X²-Ala-X³-Lys-Lys-Tyr-Leu-Asn-Ser-
Ile-Leu-Asn

wherein X¹, X² and X³ are the same or different and each is the residue of a natural or non-natural amino acid, provided that when both X¹ and X³ are valine, X² may not be methionine.

According to this embodiment, X¹, X² and X³ are the same or different and each may be selected from the group consisting of leucine, isoleucine, norleucine (Nle), valine, tryptophan, phenylalanine, methionine, octahydroindole-2-carboxylic acid, cyclohexylglycine and cyclopentylglycine.

According to another embodiment, VIP-related peptide is a conjugate of VIP or of an analogue thereof coupled to a lipophilic moiety, of the sequence (SEQ ID NO:2):

```
            1 7
R¹-Y¹-His-Ser-Asp-Ala-X¹-Phe-Thr-Asp-Asn-Tyr-Thr-
                                16
Arg-Leu-Arg-Lys-Gln-X²-Ala-X³-Lys-Lys-Tyr-Leu-Asn-
                        28
Ser-Ile-Leu-Asn-NH-Y²-R²
```

wherein X¹, X² and X³ are the same or different and each is the residue of a natural or non-natural amino acid;
R¹ and R² are the same or different and each is hydrogen, a saturated or unsaturated lipophilic group or a C₁-C₄ hydrocarbyl or C₁-C₄ carboxylic acyl, with the proviso that at least one of R¹ and R² is a lipophilic group; and

Y¹ and Y² may be the same or different and each is —CH₂— or a bond in case the associated R¹ and R² is hydrogen and Y¹ may further be —CO—.

Examples of said lipophilic group include, but are not limited to, a saturated or unsaturated carboxylic acyl having at least 5 carbon atoms selected from caproyl (Cap), lauroyl (Lau), palmitoyl, stearoyl (St), oleyl, eicosanoyl, docosanoyl, and the corresponding hydrocarbyl radicals hexyl, dodecyl, hexadecyl, octadecyl, eicosanyl, and docosanyl.

Preferred conjugates according to this embodiment of the invention include Stearoyl-VIP (St-VIP), Caproyl-norleucine¹⁷-VIP (Cap-Nle¹⁷-VIP), Stearoyl-leucine⁵, norleucine¹⁷ (St-Leu⁵, Nle¹⁷-VIP), Stearoyl-leucine⁵, leucine¹⁷ (St-Leu⁵, Leu¹⁷-VIP), Stearoyl-threonine⁷ (St-Thr⁷-VIP), and, more preferably, Stearoyl-norleucine¹⁷-VIP (St-Nle¹⁷-VIP, also designated herein SNV).

In another embodiment, the VIP-related peptide of the invention is a physiologically active fragment of VIP or a conjugate thereof with a lipophilic group selected from the group consisting of VIP₇₋₂₈, St-VIP₁₆₋₂₈, St-VIP₇₋₂₈ and St-VIP_{6-28.}

In still another embodiment, the VIP-related peptide of the invention is a physiologically active fragment of VIP or an analogue of said fragment or a conjugate of said fragment or of said fragment analogue with a lipophilic group such as, for example, the following peptides and conjugates:

| | |
|---|---|
| Ala-Val-Lys-Lys-Tyr-Leu-Asn-Ser-Ile-Leu-Asn-NH₂ | (SEQ ID NO: 3) |
| Lys-Lys-Tyr-Leu-NH₂ | (SEQ ID NO: 4) |
| Lys-Lys-Tyr-dAla-NH₂ | (SEQ ID NO: 5) |
| Val-Lys-Lys-Tyr-Leu-NH₂ | (SEQ ID NO: 6) |
| Ala-Val-Lys-Lys-Tyr-Leu-NH₂ | (SEQ ID NO: 7) |
| Asn-Ser-Ile-Leu-Asn-NH₂ | (SEQ ID NO: 8) |
| Lys-Lys-Tyr-Val-NH₂ | (SEQ ID NO: 9) |
| Ser-Ile-Leu-Asn-NH₂ | (SEQ ID NO: 10) |
| Asn-Ser-Tyr-Leu-Asn-NH₂ | (SEQ ID NO: 11) |
| Asn-Ser-Ile-Tyr-Asn-NH₂ | (SEQ ID NO: 12) |
| Ala-Val-Lys-NH₂ | (SEQ ID NO: 13) |
| Lys-Tyr-Leu-NH₂ | (SEQ ID NO: 14) |
| Lys-Lys-Tyr-Nle-NH₂ | (SEQ ID NO: 15) |
| Ala-Val-Lys-Lys-Tyr-NH₂ | (SEQ ID NO: 16) |
| Val-Lys-Lys-Tyr-Leu-NH₂ | (SEQ ID NO: 17) |
| Leu-Asn-Ser-Ile-Asn-NH₂ | (SEQ ID NO: 18) |
| Tyr-Leu-Asn-Ser-Ile-Asn-NH₂ | (SEQ ID NO: 19) |

and conjugates of said fragments with a lipophilic group such as stearoyl, caproyl and lauroyl.

With regard to the conjugates of the VIP fragments, the present invention encompasses all those conjugates disclosed in PCT Publication WO 97/40070, herein incorporated by reference in its entirety as if fully disclosed herein.

In another aspect, the invention relates to the use of a VIP-related peptide as defined above for the preparation of a pharmaceutical composition for the treatment of female sexual dysfunction and/or for vaginal relaxation, or for modulation of sperm motility.

In still another aspect, the invention provides a method for noninvasive treatment of female sexual dysfunction and/or vaginal relaxation by administration of a VIP-related peptide as described above.

The invention further provides methods to enhance or reduce sperm motility and hence copulation efficiency, both in vitro and in vivo.

### DESCRIPTION OF THE FIGURES

FIG. 1 shows that 0.3 μM VIP neutralizes vaginal contraction.

FIG. 2 shows that 0.3 μM SNV attenuated the rate of vaginal contraction stimulated by 0.5 μM phenylephrine. In addition, SNV administration also reduced the contraction force (negative inotropic effect).

FIG. 3 shows that 1.5 μM SNV blocked vaginal contraction stimulated by 0.5 μM phenylephrine.

### DETAILED DESCRIPTION OF THE INVENTION

The present invention provides according to a first of its aspects, pharmaceutical compositions, in particular, topical formulations of small VIP-related peptides including conjugates thereof with lipophilic groups, for noninvasive treatment of female sexual dysfunction and/or for vagina/relaxation.

The aspect of the vaginal relaxation is of interest in cases of pregnant women prone to abortion. This treatment is intended to prevent abortion in such women.

The compositions of the invention are intended for topical vaginal, vulvar or clitoral administration. The “pharmaceutically acceptable carrier” of the pharmaceutical composition is a material suitable for vaginal, vulvar or clitoral delivery that is non-toxic and does not affect negatively the active ingredient or any other component of the formulation. The carrier is preferably selected from amongst those which enhance the tissue penetration of the active ingredient and include, without being limited to, carriers such as glycerine, lubricants, olive oil, nitroglycerine, glyceryl monocaprylate, propylene glycol didecanoate, propylene glycol dicaprylate, glyceryl tricaprylate, sorbitan monocaprylate, and mixtures thereof.

Other compositions suitable for local administration are as previously described (Okada et al., J. Pharmaceut. Sci. 71, 1367-1371, 1982) and may include jelly, starch, or protease inhibitors, or Sefsol™/isopropanol (Gozes et al., Endocrinology, 134,2121-2125,1994). Pharmaceutical creams, viscous liquid or semi-solid emulsion containing oil phase and water-based phase can be prepared as described in Remington's Pharmaceutical Sciences, 18^{th} Ed., Easton, Pa.: Mack Publishing Company, 1990. Other suitable form for drug delivery is as liposomes (microscopic vesicles having a lipid wall comprising a lipid bilayer, e.g. lipofactin, GIBCO, BRL, Grand Island, N.Y.).

An effective amount of the pharmaceutically active ingredient of the invention is an amount that is sufficient to provide the desired degree of treatment.

The invention is preferably applied to human females, although it can also be applied to other species, such as bovine, canine, equine, ovine and porcine.

The composition is preferably locally administered to a portion of the wall of the female genital tract, most preferably to a portion of the inner wall of the vagina.

Local administration can be accomplished by diffusion from a solution dispersed in to a suitable support, such as a porous tampon, a suppository made with a composition comprising oleaginous base materials, or a suitable composition such as emulsion, cream, jelly, or tablet or using a applicator such as applicators used to self-administer contraceptive foam.

The invention will now be illustrated by the following non-limitative Examples.

### EXAMPLE 1

Synthesis of the VIP-Related Peptides

The VIP-related peptides used in the compositions of the invention are prepared by standard peptide synthesis procedures well known in the art. The conjugates with a lipophilic group are prepared substantially as described in U.S. Pat. No. 5,147,855, U.S. Pat. No. 5,998,368, EP 620008, and PCT Publication WO 97/40070, all these documents being herein incorporated by reference in their entirety as if fully disclosed herein.

### EXAMPLE 2

Cream and Ointment Formulations

Cream and ointment formulations comprising a VIP-related peptide such as, for example, stearoyl-VIP, stearoyl-Nle¹⁷VIP (SNV), Lys-Lys-Tyr-Leu ((SEQ ID NO:20)or Asn-Ser-Ile-Leu-Asn (SEQ ID NO:21), are prepared substantially as described in U.S. Pat. No. 5,877,216, U.S. Pat. No. 5,998,368, and PCT WO 97/40070.

The concentration of the VIP-related peptide in the physiologically acceptable solution is between 0.01 micrograms and about 100 micrograms per ml.

### EXAMPLE 3

Candidate sexual dysfunction patients are assembled from a group of female individuals assessed and prescreened. Following topical administration of a composition of the invention to the vagina, vulvar area or clitoris, changes in uterine or vaginal epithelial blood flow are measured by known methods. Increase in vaginal epithelial blood flow is measured by photoplethysmography (Levin et al., 1980, Clinics in Obstet. Gynaecol. 7: 213-252), heated oxygen electrode (Wagner et al., 1978, “Vaginal Fluid” in The Human Vagina, Evans et al. (eds), Amsterdam, Elsevier, North Holland Biomedical Press, pp. 121-137), and direct clearance of radioactive Xenon (Wagner et al., 1980, Obstet. Gynaecol. 56: 621-624). Changes in vulvar blood flow are measured using Doppler velocimetry (U.S. Pat. No. 5,877,216, Australian Patent 609765), and increased lubrication is measured using pre-weighed circular filter papers arranged in a layer and held inside a plastic suction capsule and in direct contact with the vaginal wall, additional fluid is measured by weight at determined times after application (Australian Patent 609765).

### EXAMPLE 4

Smooth muscle tone regulation in rabbit cavernosal and spongiosal tissue follows the method described by Spawasser et al., J. Urology 152,2159-2163, 1994. Briefly, rabbit is used as a model (Taub et al., Urology, 42,698-704, 1993), and longtitudinal strips (cavernous and urethral) are obtained, and mounted between two metal hooks in an organ bath chamber containing 10 ml of Krebs solution. Tension is measured by an adjustable connection to a Grass Instrument force-displacement transducer. Strips are preconditioned with phenylephrine at a concentration of 3×10⁶ mol./l (40% maximal contraction). Control uses papaverine (Chen, K.-K., et al., J. Urology 147,1124-1128, 1992; Spawasser et al. 1994, ibid), alprostadil and VIP 1-28. All experiments comprise dose-response curves. Similar experiments are conducted with female rabbit vaginal and clitorial smooth muscle (Ottesen et al., 1983, ibid), or utilizing a rabbit clitorial smooth muscle cell culture to study receptor binding and direct effects (H. Sadehi-Nejad et al., International J. Imp. Res. 10, 165-169, 1998). Blood flow is measured in a rabbit animal model as described before (Park et al., International J. Imp. Res. 9, 27-37, 1997).

### EXAMPLE 5

Cyclic GMP has been associated with smooth muscle relaxation and hence directly implicated in sexual arousal and increased blood flow to the sex organs. SNV is 1000-fold more potent than VIP in inducing cGMP formation (Ashur-Fabian 1999, ibid). Other VIP-related peptides (Gozes et al., 1999 ibid) are tested in a similar assay system.

### EXAMPLE 6

Stimulation/inhibition of sperm motility (Gozes et al., 1998 ibid), stimulation/inhibition of sperm maturation (Golan et al., Mol Reprod Dev. 56, 105-112, 2000) and zygote formation (Ravhon et al., Fertil Steril. 73, 908-912, 2000) are tested with the VIP-related peptides as described in said references, all being herein incorporated by reference in their entirety.

### EXAMPLE 7

The 28 amino acid, vasoactive intestinal peptide (VIP) may induce the physiological changes in sexual arousal and excitement, and may be the major neurotransmitter that participates in the innervation of the vaginal blood supply, including small blood vessels, smooth muscle and epithelial cells in the vaginal tract (U.S. Pat. No. 5,877,216; Australian patent 609765). Systemic administration of VIP to females has been found to decrease uterine smooth muscle activity and increase vaginal blood flow (Ottesen et al., Eur. J. Clin. Invest. 13, 321-324,1983). In males, VIP induces penile erection and is thus suggested to induce clitorial arousal in the female (Hauser-Kronbrger et al., Peptides 20, 539-543, 1999). VIP derivatives and conjugates were designed to include a lipophilic moiety or a shortened VIP chain (Gozes and Fridkin, J. Clin. Invest. 90, 810-814, 1992; Gozes et al., Endocrinology, 134,2121-2125,1994; Gozes et al., J. Pharmacol. Exper. Therap. 273, 161-167, 1995; Gozes et al., Proc. Natl. Acad. Sci. USA 93, 427-432, 1996; Gozes et al., Proc. Natl. Acad. Sci. USA 96, 4143-4148, 1999). These derivatives may distinguish different VIP receptors (Gozes et al., 1995 ibid.) and enhance cGMP formation, 1000-fold more potently than VIP (Ashur-Fabian et al., Peptides 20, 629-633, 1999). Interestingly, the proposed mechanism of action of Sildenafil Citrate is through inhibition of cGMP breakdown (Moreland et al., Trends Endocrinol Metab. 10, 97-104, 1999). Given the proposed mechanism of action (Gozes and Fridkin, 1992, ibid.; Gozes et al., 1994 ibid.; Gozes et al., 1995, ibid.; Gozes et al., 1996, ibid.; Gozes et al., 1999, ibid.; Ashur-Fabian et al., 1995, ibid) and the increased bioavailability (Gozes and Fridkin, 1992, ibid.; Gozes et al., 1994, ibid.), lipopophilic VIP derivatives are now suggested as potential topical treatment for female sexual dysfunction. The first compound chosen for further investigations was Stearoyl-Nle¹⁷VIP=SNV, comprising the VIP molecule with two modifications, namely an N-terminal attachment of stearic acid residue and an exchange of the methionine residue in position 17 with norleucine. SNV exhibits increased stability and bioavailability (Gozes et al., 1994, ibid.). SNV was now tested for its ability to induce vaginal relaxation.

Methods:

The vagina of 12-week-old New Zealand female rabbit was removed, exiced to 1 cm length and immersed in 10 ml of capacity organ bath connected to an external reservoir of Krebs-Henseleit in constant temperature of 37° C. The solution was oxygenated (95%O₂/25% CO₂). The effect of VIP and SNV on vaginal contraction was measured by attaching the vaginal muscle to a force transducer.

Results:

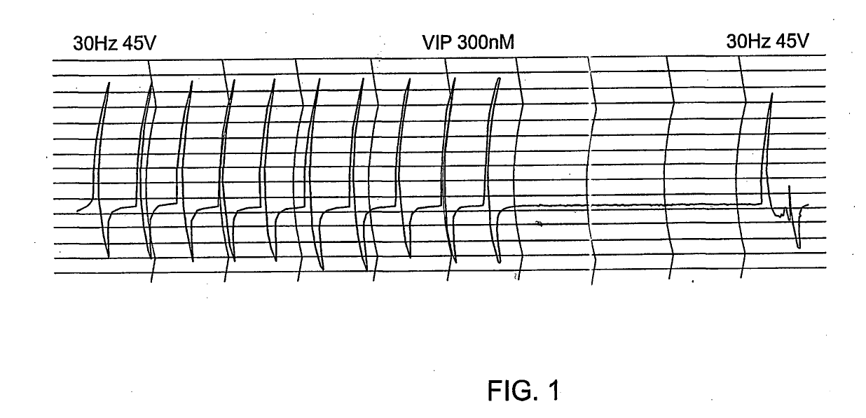

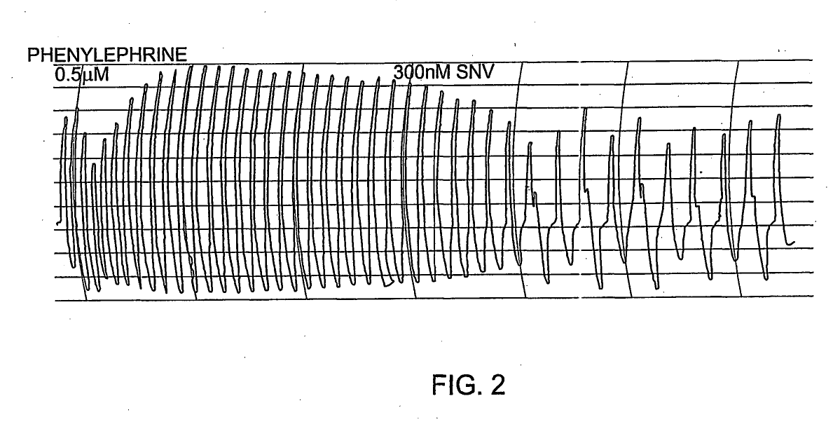

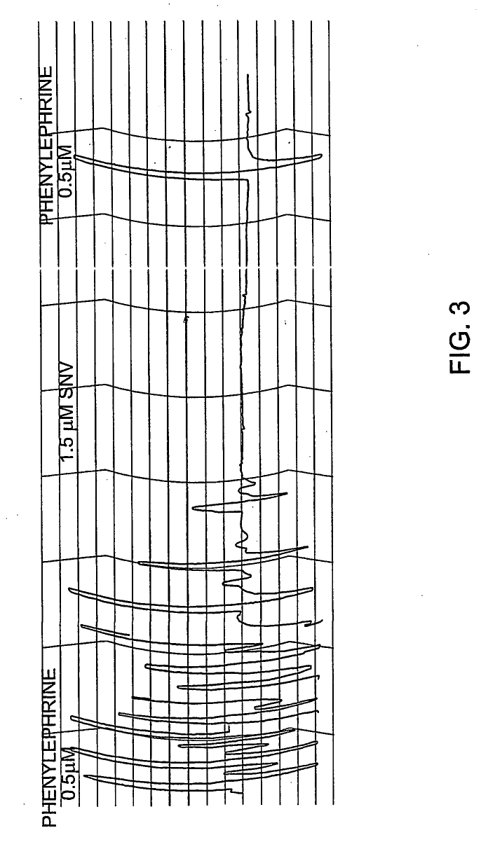

Preliminary results showed that 0.3 μM of VIP neutralized vaginal contraction (FIG. 1). At the same concentration, SNV attenuated the rate of contraction, stimulated by 0.5 μM phenylephrine (FIG. 2). In addition, SNV administration also reduced the contraction force (negative inotropic effect). High concentrations of SNV (1.5 M) neutralized vaginal contraction stimulated by 0.5 PM phenylephrine (FIG. 3).

From these experiments we can conclude that SNV may protect against excessive vaginal dilatation and that SNV holds promise for the treatment of female sexual dysfunction.

1-36. (canceled)

37. A method for the treatment of female sexual dysfunction and/or for vaginal relaxation, comprising administering to a female individual a pharmaceutical composition comprising an effective amount of a peptide selected from:
(i) a peptide analogue of vasoactive intestinal peptide (VIP) in which one or more amino acids has been replaced, added or deleted without substantially altering the biological properties of the parent peptide;
(ii) a conjugate of VIP or of a peptide analogue of (i) coupled to a lipophilic moiety;
(iii) a physiologically active fragment of VIP, or of a peptide analogue
(i) or of a conjugate (ii); and
(iv) a functional derivative of any of (i), (ii) and (iii).

38. The method of claim 37, for improving vaginal muscle tone and vaginal tissue health, for enhancing vaginal lubrication and enhancing sexual arousal.

39. The method of claim 37, wherein said pharmaceutical composition is for topical application in the vaginal, vulvar and/or clitorial area.

40. The method of claim 39, wherein pharmaceutical composition is in the form of a cream, gel, suspension, ointment, solution, foam or liposomal composition.

41. The method of claims 37, wherein said analogues of vasoactive intestinal peptide (VIP) in which one or more amino acids has been replaced have the sequence:
```
1 7
His-Ser-Asp-Ala-X¹-Phe-Thr-Asp-Asn-Tyr-Thr-Arg-
                        16
Leu-Arg-Lys-Gln-X²-Ala-X³-Lys-Lys-Tyr-Leu-Asn-Ser-
                28
Ile-Leu-Asn
```
wherein X¹, X² and X³ are the same or different and each is the residue of a natural or non-natural amino acid, provided that when both X¹ and X³ are valine, X² may not be methionine.

42. The method of claim 41, wherein X¹, X² and X³ are the same or different and each is selected from leucine, isoleucine, norleucine (Nle), valine, tryptophan, phenylalanine, methionine, octahydroindole-2-carboxylic acid, cyclohexylglycine and cyclopentylglycine.

43. The method of claim 37, wherein said conjugates of VIP or analogues thereof coupled to a lipophilic moiety; have the sequence:
```
            1 7
R¹-Y¹-His-Ser-Asp-Ala-X¹-Phe-Thr-Asp-Asn-Tyr-Thr-
                                16
Arg-Leu-Arg-Lys-Gln-X²-Ala-X³-Lys-Lys-Tyr-Leu-Asn-
                    28
Ser-Ile-Leu-Asn-NH-Y²-R²
```
wherein X¹, X² and X³ are the same or different and each is the residue of a natural or non-natural amino acid;
R¹ and R² are the same or different and each is hydrogen, a saturated or unsaturated lipophilic group or a C₁-C₄ hydrocarbyl or C₁-C₄ carboxylic acyl, with the proviso that at least one of R¹ and R² is a lipophilic group; and
Y¹ and Y² may be the same or different and each is —CH₂— or a bond in case the associated R¹ and R² is hydrogen and Y¹ may further be —CO—.

44. The method claim 43, wherein said lipophilic group of said conjugate is a saturated or unsaturated carboxylic acyl having at least 5 carbon atoms selected from caproyl (Cap), lauroyl (Lau), palmitoyl, stearoyl (St), oleyl, eicosanoyl, docosanoyl, and the corresponding hydrocarbyl radicals hexyl, dodecyl, hexadecyl, octadecyl, eicosanyl, and docosanyl.

45. The method of claim 44, wherein said conjugate is selected from:
Stearoyl-VIP (St-VIP)
Stearoyl-norleucine¹⁷-VIP (St-Nle⁷-VIP)
Caproyl-norleucine¹⁷-VIP (Cap-Nle¹⁷-VIP)
Stearoyl-leucine⁵, norleucine¹⁷ (St-Leu⁵, Nle¹⁷-VIP)
Stearoyl-leucine³, leucine¹⁷ (St-Leu⁵, Leu¹⁷-VIP)
Stearoyl-threonine⁷ (St-Thr⁷-VIP).

46. The method of claim 37, wherein said physiologically active fragments of VIP and conjugates thereof with a lipophilic group are selected from VIP₇₋₂₈, St-VIP₁₆₋₂₈, St-VIP₇₋₂₈ and St-VIP₁₆₋₂₈.

47. The method of claim 37, wherein said physiologically active fragments of VIP or of analogues thereof are selected from:
Ala-Val-Lys-Lys-Tyr-Leu-Asn-Ser-Ile-Leu-Asn-NH₂
Lys-Lys-Tyr-Leu-NH₂
Lys-Lys-Tyr-dAla-NH₂
Val-Lys-Lys-Tyr-Leu-NH₂
Ala-Val-Lys-Lys-Tyr-Leu-NH₂
Asn-Ser-Ile-Leu-Asn-NH₂
Lys-Lys-Tyr-Val-NH₂
Ser-Ile-Leu-Asn-NH₂
Asn-Ser-Tyr-Leu-Asn-NH₂
Asn-Ser-Ile-Tyr-Asn-NH₂
Ala-Val-Lys-NH₂
Lys-Tyr-Leu-NH₂
Lys-Lys-Tyr-Nle-NH₂
Ala-Val-Lys-Lys-Tyr-NH₂
Val-Lys-Lys-Tyr-Leu-NH₂
Leu-Asn-Ser-Ile-Asn-NH₂
Tyr-Leu-Asn-Ser-Ile-Asn-NH₂
and conjugates of said fragments with a lipophilic group selected from stearoyl, caproyl and lauroyl.

48. A method for modulation of sperm motility in a female individual, comprising administering to said female individual a pharmaceutical composition comprising a peptide selected from:
(i) a peptide analogue of vasoactive intestinal peptide (VIP) in which one or more amino acids has been replaced, added or deleted without substantially altering the biological properties of the parent peptide;
(ii) a conjugate of VIP or of a peptide analogue of (i) coupled to a lipophilic moiety;
(iii) a physiologically active fragment of VIP, or of a peptide analogue (i) or of a conjugate (ii); and
(iv) a functional derivative of any of (i), (ii) and (iii).

49. The method of claim 48, wherein said pharmaceutical composition is for topical application in the vaginal, vulvar and/or clitorial area.

50. The method of claim 49, wherein pharmaceutical composition is in the form of a cream, gel, suspension, ointment, solution, foam or liposomal composition.

51. The method of claim 48, wherein said analogues of vasoactive intestinal peptide (VIP) in which one or more amino acids has been replaced have the sequence:
```
1 7
His-Ser-Asp-Ala-X¹-Phe-Thr-Asp-Asn-Tyr-Thr-Arg-
                        16
Leu-Arg-Lys-Gln-X²-Ala-X³-Lys-Lys-Tyr-Leu-Asn-Ser-
                28
Ile-Leu-Asn
```
wherein X¹, X² and X³ are the same or different and each is the residue of a natural or non-natural amino acid, provided that when both X¹ and X³ are valine, X² may not be methionine.

52. The method of claim 51, wherein X¹, X² and X³ are the same or different and each is selected from the group consisting of leucine, isoleucine, norleucine (Nle), valine, tryptophan, phenylalanine, methionine, octahydroindole-2-carboxylic acid, cyclohexylglycine and cyclopentylglycine.

53. The method of claim 48, wherein said conjugates of VIP or analogues thereof coupled to a lipophilic moiety; have the sequence:
```
            1 7
R¹-Y¹-His-Ser-Asp-Ala-X¹-Phe-Thr-Asp-Asn-Tyr-Thr-
                                16
Arg-Leu-Arg-Lys-Gln-X²-Ala-X³-Lys-Lys-Tyr-Leu-Asn-
                    28
Ser-Ile-Leu-Asn-NH-Y²-R²
```
wherein X¹, X² and X³ are the same or different and each is the residue of a natural or non-natural amino acid;
R¹ and R² are the same or different and each is hydrogen, a saturated or unsaturated lipophilic group or a C₁-C₄ hydrocarbyl or C₁-C₄ carboxylic acyl, with the proviso that at least one of R¹ and R² is a lipophilic group; and
Y¹ and Y may be the same or different and each is —CH₂— or a bond in case the associated R¹ and R² is hydrogen and Y¹ may further be —CO—.

54. The method of claim 53, wherein said lipophilic group of said conjugate is a saturated or unsaturated carboxylic acyl having at least 5 carbon atoms selected from caproyl (Cap), lauroyl (Lau), palmitoyl, stearoyl (St), oleyl, eicosanoyl, docosanoyl, and the corresponding hydrocarbyl radicals hexyl, dodecyl, hexadecyl, octadecyl, eicosanyl, and docosanyl.

55. The method of claim 54, wherein said conjugate is selected from:
Stearoyl-VIP (St-VIP)
Stearoyl-norleucine¹⁷-VIP (St-Nle¹⁷-VIP)
Caproyl-norleucine¹⁷-VIP (Cap-Nle¹⁷-VIP)
Stearoyl-leucine⁵, norleucine¹⁷ (St-Leu⁵, Nle¹⁷-VIP)
Stearoyl-leucine⁵, leucine¹⁷ (St-Leu⁵, Leu¹⁷-VIP)
Stearoyl-threonine⁷ (St-Thr⁷-VIP)

56. The method of claim 48, wherein said physiologically active fragments of VIP and conjugates thereof with a lipophilic group are selected from VIP₇₋₂₈, St-VIP₁₆₋₂₈, St-VIP₇₋₂₈ and St-VIP₁₆₋₂₈.

57. The method of claim 48, wherein said physiologically active fragments of VIP or of analogues thereof are selected from:
Ala-Val-Lys-Lys-Tyr-Leu-Asn-Ser-Ile-Leu-Asn-NH₂
Lys-Lys-Tyr-Leu-NH₂
Lys-Lys-Tyr-dAla-NH₂
Val-Lys-Lys-Tyr-Leu-NH₂
Ala-Val-Lys-Lys-Tyr-Leu-NH₂
Asn-Ser-Ile-Leu-Asn-NH₂
Lys-Lys-Tyr-Val-NH₂
Ser-Ile-Leu-Asn-NH₂
Asn-Ser-Tyr-Leu-Asn-NH₂
Asn-Ser-Ile-Tyr-Asn-NH₂
Ala-Val-Lys-NH₂
Lys-Tyr-Leu-NH₂
Lys-Lys-Tyr-Nle-NH₂
Ala-Val-Lys-Lys-Tyr-NH₂
Val-Lys-Lys-Tyr-Leu-NH₂
Leu-Asn-Ser-Ile-Asn-NH₂
Tyr-Leu-Asn-Ser-Ile-Asn-NH₂
and conjugates of said fragments with a lipophilic group selected from stearoyl, caproyl and lauroyl.
